(19) United States
(12) Patent Application Publication
(10) Pub. No.: US 2021/0093552 A1
(43) Pub. Date: Apr. 1, 2021

(54) INHALABLE CANNABINOID COMPOSITIONS AND USES

(71) Applicant: SENZER LIMITED, London (GB)

(72) Inventors: Alex HEARN, London (GB); Rene M. GONZALEZ, London (GB); Paul YOUNG, Essex (GB); Ritika GUPTA, London (GB)

(21) Appl. No.: 16/710,148

(22) Filed: Dec. 11, 2019

(30) Foreign Application Priority Data
Sep. 27, 2019 (GB) 1913956.7

(51) Int. Cl. A61K 9/00; A61K 31/05; A61K 31/352; A61K 47/10; A61K 47/46; A61K 9/12; A61M 15/00

(52) U.S. Cl. CPC A61K 9/008; A61K 31/05; A61K 31/352; A61M 15/002; A61K 47/46; A61K 9/12; A61M 15/0013; A61K 47/10

(57) ABSTRACT

An inhalable composition comprising: one or more cannabinoids or a pharmaceutically acceptable derivative or salt thereof; a propellant comprising HFA-152a; and a glycol and/or glycol ether.

### CROSS-REFERENCE TO RELATED APPLICATION

This Application claims priority from Application 1913956.7 filed on Sep. 27, 2019 in the United Kingdom, the contents of which are herein incorporated by reference.

### BACKGROUND

Cannabinoids have long been known for their therapeutic potential in pain relief, treatment of seizures, antiemesis etc. It is, however, a class of compounds whose usage has been associated with a great deal of debate owing to its psychoactive effects. It was not until the discovery of cannabinoid receptors (CB1 and CB2) and the isolation of individual cannabinoids such as THC (tetrahydrocannabinol), CBD (cannabidiol), CBN (Cannabinol), and THCV (Tetrahydrocannabivarin), that the psychoactive effects could be attributed primarily to compounds (like THC) with high affinities to the receptor CB1. Furthermore, it has been established that individual cannabinoids differ from one another in their affinities to receptors and certain cannabinoids, such as CBD, behave as CB1/CB2 antagonists, thereby blocking some actions of their agonists, such as THC.

With on-going research, therapeutic applications of cannabinoids are becoming increasingly evident, resulting in legalisation of these compounds for medical purposes in a number of countries. The primary targets of research in this field are being associated with safe, rapid and/or effective delivery of cannabinoids.

A number of ways of delivering cannabinoids are known in the art.

For example, US2012/0304990 teaches the use of heating to vaporise a cannabis deposit. One drawback of this system is that there is only a small temperature differential between the temperature at which the cannabis will vaporise (180° C. to 200° C.) and the temperature at which toxins are produced (230° C.)

A number of documents (for example WO03/055549, U.S. Pat. No. 6,509,005 and WO2004/000290) disclose the use of a metered dose inhaler. Such inhalers suffer from a number of drawbacks. Firstly, the metering chamber is relatively small, generally less than 100 μl resulting in delivery of fairly concentrated doses. Also, such devices require users to optimally co-ordinate actuation of the outlet valve and inhalation, failing which, dose delivery could be variable.

A further common mechanism is the simple spray, which is disclosed, for example, in WO02/064109 and US2006/135599, which are designed to provide a sublingual or buccal spray. Such a spray is currently being marketed by GW Pharmaceuticals under the Sativex (™) brand. These sprays suffer from the possibility of non-uniform drug dose delivery owing to the flushing action of saliva. Further, they have a slower onset of action when compared with pulmonary delivery.

WO0166089 describes a mode of administration for cannabis and its natural and synthetic derivatives. A pharmaceutical composition suitable for sublingual aerosol or spray delivery of cannabis is provided. The formulation may be dispensed using a pump spray or the formulation may include a propellant, such as butane, 1,1,1,2-tetrafuorethane (HFC-134a) or 1,1,1,2,3,3,3-heptafluropropane (HFC-227). WO03055549 describes a medicinal aerosol product comprising a pressurized metered dose inhaler, including a canister equipped with a metering valve and containing a medicinal aerosol solution formulation, and an actuator comprising a nozzle block defining an actuator orifice leading to an expansion chamber, wherein the formulation includes a cannabinoid, a hydrofluorocarbon propellant and an optional amount of an alcohol co-solvent, and the actuator orifice has a diameter of about 0.30 mm or less, and/or is laser drilled. WO2007002186 describes pharmaceutical compositions comprising delta-9-tetrahydrocannabinol and methods of administering such compositions to treat migraines. WO0113886 describes a formulation of delta-9-tetrahydrocannabinol in a semi-aqueous solvent, such as 35:10:55 alcohol:water:propylene glycol (v/v), produces a stable clear solution near the solubility point of the drug. Because delta-9-tetrahydrocannabinol has poor affinity for the formulation, it is able to partition out and transport across cell membranes to reach the bloodstream quickly. This has been demonstrated by the comparative tmax values achieved in single dose intravenous and 14 day multiple dose inhalation studies conducted in dogs and rats. WO2004000290 describes a pharmaceutical composition for administration as an aerosol, which comprises a cannabinoid, a propellant and an effective amount of a cough suppressant. WO2011107737 describes a simulated cigarette device containing a pressurised reservoir of inhalable composition and an outlet valve controlling the output of the inhalable composition. A capillary plug extends from the vicinity of the outlet to fill a substantial portion of the reservoir and is configured to wick the inhalable composition towards the outlet valve.

WO2015/121673 discloses an inhalable cannabinoid composition. While such compositions may be delivered to a user with a favourable droplet profile, such compositions contain alcohol. Accordingly, the compositions may be less suitable for users who are adverse to the consumption of alcohol for medical or religious reasons. Furthermore, the propellants disclosed therein have a significant “carbon footprint”.

The present disclosure seeks to tackle at least some of the problems associated with the prior art or at least to provide a commercially acceptable alternative solution thereto.

### SUMMARY

In a first aspect, the present invention provides an inhalable composition comprising:
one or more cannabinoids or a pharmaceutically acceptable derivative or salt thereof;
a propellant comprising HFA-152a; and
a glycol and/or glycol ether.

Each aspect or embodiment as defined herein may be combined with any other aspect(s) or embodiment(s) unless clearly indicated to the contrary. In particular, any features indicated as being preferred or advantageous may be combined with any other feature indicated as being preferred or advantageous.

The inventors have surprisingly found the inhalable composition may exhibit increased stability in comparison to conventional cannabinoid-containing inhalable compositions. Furthermore, in comparison to conventional cannabinoid-containing inhalable compositions, the inhalable composition of the present invention is capable of delivering a required dose of cannabinoid to a user using lower levels of propellants and solvents.

Since the composition is inhalable, and is typically predominately absorbed in the lungs, the onset of therapeutic action is rapid. This is in contrast to cannabinoid delivery methods such as sublingual delivery, which predominantly involves slower uptake via a buccal route or the inhalation of cannabinoids vaporised from a cannabis plant surface, which requires high temperatures. In comparison to delivery methods such as suppository delivery and sublingual delivery, the composition of the present invention may provide a uniform and reliable drug absorption profile. The composition may also be administered in a convenient and hygienic manner.

The glycol and/or glycol ether may aid the dissolution of the one or more cannabinoids or pharmaceutically acceptable derivatives or salts thereof in the composition. This avoids the presence of precipitates of cannabinoids (or other additives such as nicotine and/or saccharin, if present) in the composition, which could cause irritation when delivered to a user. In addition, the presence of glycol or glycol ether reduces the degradation of the cannabinoids that may occur over time, thereby increasing the long-term stability or “shelf life” of the composition.

The propellant comprises HFA-152a, i.e. 1,1-difluoroethane (sometime referred to as R-152a). Typically, the propellant consists essentially of HFA-152a, more typically the propellant consists of HFA-152a. In comparison to propellants used in conventional cannabinoid-containing inhalable compositions, it has surprisingly been found that HFA-152a; is better able to solubilise cannabinoid compounds. Accordingly, the one or more cannabinoids or a pharmaceutically acceptable derivative or salt thereof is more likely to remain in solution before or during use. This may result in a longer shelf-life of the composition. In addition, this may ensure that a higher proportion of the one or more cannabinoids or a pharmaceutically acceptable derivative or salt thereof is delivered to the lungs rather than, for example, the oropharynx.

Advantageously, the increased solubility of cannabinoids in HFA-152a means that the composition requires only low levels of alcohol (e.g. ethanol), typically substantially no alcohol, to solubilise the one or more cannabinoids or a pharmaceutically acceptable derivative or salt thereof. In contrast, the inventors have found that in an alcohol-free cannabinoid-containing composition containing HFA-134a (a conventional propellant for cannabinoid compositions), the cannabinoid is inadequately solubilised. Accordingly, the inhalable composition of the present invention may be advantageous for users adverse to the consumption of alcohol, for example on medical or religious grounds. Furthermore, alcohol-free compositions may result in less dryness and/or irritation to body tissues to which they come into contact during use.

Since the HFA-152a acts as both a propellant and a solvent, the inhalable composition may be simpler and cheaper to manufacture than conventional inhalable compositions due to the fact that it contains fewer components.

In comparison to propellants used in conventional inhalable cannabinoid compositions, HFA-152a exhibits a lower carbon footprint. It has an ozone depletion potential of zero, a low global warming potential (124) and a short atmospheric lifetime (1.4 years). For example, the carbon footprint of HFA-152a is reported to be around 12 times lower than that of the conventional propellant HFA-134a.

Advantageously, when a cannabinoid composition having such a combination of HFA-152a and glycol or glycol ether is delivered to a user via a conventional pressurised metered-dose inhaler (pMDI), the composition is delivered in the form of droplets, some of which (such as, for example, at least 10% vol) have a diameter of less than 10 μm, typically less than 5 μm. The term “diameter” as used herein encompasses the largest dimension of a droplet, and may be measured using a Malvern Spraytec device. Typically, the majority (such as, for example, at least 50% vol) of the droplets have a diameter of less than 5 μm, typically substantially all (such as, for example, at least 90% vol, or even at least 95% vol) of the droplets have a diameter of less than 5 μm. Advantageously, when administered to a user, droplets with a size of less than 10 μm tend to be deposited in the lungs, rather than, for example, the oropharynx. Accordingly, at least some (such as, for example, at least 10% w/w), typically substantially all (such as, for example, at least 90% w/w), of the cannabinoid enters the bloodstream via the pulmonary route, which results in rapid absorption of the composition.

Typically at least some (such as, for example, at least 10% vol) of the droplets have a size of from 0.5 to 3 μm. Such droplets may be deposited in the deep lung, and are therefore particularly able to enter the blood stream via the pulmonary route. Typically at least some (such as, for example, at least 10% vol) of the droplets have a diameter of from 0.4 to 0.5 μm.

In contrast to compositions of the prior art, the composition of the present invention is able to form small diameter droplets without the use of alcohols (e.g. ethanol) or organic acids. Accordingly, the level of irritation experienced by a user of the compositions is reduced.

When the composition of the present invention is delivered to a user via one of the inhalers described herein, the droplets may exhibit the following droplet size profile:

Dv 90 of less than 20 μm, typically less than 10 μm, more typically less than 8, even more typically less than 6 μm, and/or

Dv 50 of less than 6 μm, typically less than 4 μm, more typically less than 3 μm, even more typically less than 1 μm, and/or

Dv 10 of less than 4 μm, typically less than 2 μm, more typically less than 1 μm, even more typically less than 0.5 μm.

The term “Dv10” as used herein refers to a droplet diameter that which 10% vol of the droplets in a composition have a smaller diameter. The term “Dv50” as used herein refers to a droplet diameter that which 50% vol of the droplets in a composition have a smaller diameter. The term “Dv90” as used herein refers to a droplet diameter that which 90% vol of the droplets in a composition have a smaller diameter. Dv10, Dv50 and Dv90 values may be determined using a Malvern Spraytec device.

The composition of the present invention may be delivered to a user via oral inhalation, specifically via pulmonary administration. Accordingly, it is effective for use in cannabis replacement therapy or as an alternative to recreational smoking of conventional cannabis-containing cigarettes, since it mimics some of the habitual aspects of cannabis smoking.

The composition preferably comprises less than 1% w/w ethanol based on the total weigh of the composition (e.g. from 0.1 to 1% w/w ethanol), more preferably less than 0.8% w/w ethanol, even more preferably less than 0.5% w/w ethanol, even more preferably less than 0.3% w/w ethanol.

In a preferred embodiment, the composition is substantially free of ethanol, or completely free of ethanol.

In another embodiment, the composition contains from 0.1 to less than 0.5% w/w ethanol. In combination with the use of HFA-152a, such low amounts of ethanol may be sufficient to solubilise the one or more cannabinoids or a pharmaceutically acceptable derivative or salt thereof, but low enough to substantially avoid the disadvantages associated with incorporating ethanol.

The term “cannabinoid” as used herein may encompass a chemical compound that activates any mammalian cannabinoid receptor, for example human CB₁ receptor or human CB₂ receptor. As used herein a chemical compound that activates a mammalian cannabinoid receptor includes agonists of said receptor. The skilled person may readily determine whether a compound is a cannabinoid receptor agonist or activator using assays known in the art, for example using a suitable [³⁵S]GTP-S binding assay (see, for example Griffin et al, Journal of Pharmacology and Experimental Therapeutics, 285(2), pp. 553-560, 1998). The cannabinoids may be naturally occurring (such as, for example, endocannabinoids or phytocannabinoids) or they may be synthetic. Synthetic cannabinoids may include, for example, the classical cannabinoids structurally related to THC, the non-classical cannabinoids (cannabimimetics) including the am inoalkyindoles, 1,5-diarylpyrazoles, quinolines and arylsulphonoam ides, and eicosanoids related to the endocannabinoids. When a cannabinoid salt is used, it may be employed in the form of a solution. The one or more cannabinoids is preferably selected from the classical cannabinoids, more preferably selected from tetrahydrocannabinols (THC), preferably delta-9-tetrahydrocannabinol and delta-8-tetrahydrocannabinol, more preferably (−)-(−)-trans-Δ⁹-tetrahydrocannabinol and trans-Δ⁸-tetrahydrocannabinol, cannabidiol (CBD), cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV and cannabigerol monomethyl ether (CBGM). CBD and THC are particularly preferred cannabinoids in the present invention. In a particularly preferred embodiment, the composition comprises both CBD and THC. CBD typically exhibits only a minimal psychoactive effect. CBD may also act as a potent antagonist to the CB1 receptor, thereby counteracting the psychoactive effect of THC without altering its advantageous clinical effects. The compound “tetrahydrocannabinol” as referred to herein may encompass (−)-(6aR, 10aR)-6,6,9-trimethyl-3-pentyl-6a, 7, 8, 10a-tetrahydro-6H-benzo[c]chromen-1-ol. The compound “cannabidiol” as referred to herein may encompass 2-[(1R,6R)-6-isopropenyl-3-methylcyclohex-2-en-1-yl]-5-pentylbenzene-1,3-diol.

Other cannabinoids suitable for use in the present invention are endocannabinoids, substances that naturally occur in the mammalian body and which activate one or more cannabinoid receptor. Preferably endocannabinoids are selected from arachidonoylethanolamine (AEA), 2-arachidonoylglycerol (2-AG), 2-arachidonyl glyceryl ether (noladin ether), N-arachidonoyl dopamine (NADA), virodhamine (OAE) and lysophosphatidylinositol (LPI).

Synthetic cannabinoids suitable for use in the present invention include nabilone, rimonabant, JWH-018, JWH-073, CP-55940, dimethylheptylpyran, HU-210, HU-331, SR144528, WIN 55,212-2, JWH-133, levonantradol, and AM-2201.

The one or more cannabinoids is preferably selected from tetrahydrocannabinol (THC), preferably dronabinol, cannabidiol (CBD), cannabinol (CBN), tetrahydrocannabivarin (THCV), cannabigerol (CBG), cannabidivarin (CBDV) and cannabichromene (CBC). Such cannabinoids may provide a particularly desirable therapeutic and/or psychoactive effect. In addition, such cannabinoids may exhibit particularly high solubility in HFA-152a.

In a particularly preferred embodiment, the one or more cannabinoids is dronabinol.

The composition preferably comprises from 0.01 to 15% w/w of the one or more cannabinoids or pharmaceutically acceptable derivatives or salts thereof based on the total weight of the composition, more preferably from 0.01 to 10% w/w, even more preferably from 0.1 to 8% w/w, still even more preferably from 1 to 5% w/w. The composition preferably comprises greater than 3% w/w of the one or more cannabinoids or pharmaceutically acceptable derivatives or salts thereof based on the total weight of the composition, more preferably greater than 4% w/w, even more preferably greater than 5% w/w. Such amounts may provide a particularly suitable therapeutic and/or psychoactive effect while substantially remaining in solution in the HFA-152a and glycol and/or glycol ether.

The glycol and/or glycol ether is preferably selected from the group consisting of propylene glycol, polypropylene glycol, polyethylene glycol (PEG), and combinations of two or more thereof. Suitable polyethylene glycols may have a molecular mass of less than 20,000 g/mol. An example of a suitable polyethylene glycol is PEG 400. Preferably, the glycol or glycol ether is propylene glycol. Propylene glycol provides the composition with a particularly desirable droplet size profile and provides enhanced solvation of excipients and reduces degradation of excipients.

Preferably, the glycol and/or glycol ether is propylene glycol and the composition comprises from 0.01 to 5% w/w propylene glycol, based on the total weight of the composition, more preferably from 0.1 to 3% w/w, even more preferably from 0.3 to 2% w/w. The composition preferably comprises at least 0.5% w/w propylene glycol, more preferably at least 0.8% w/w, even more preferably at least 1% w/w.

Preferably the composition further comprises a human TAS2R bitter taste receptor agonist. The use of a human TAS2R bitter taste receptor agonist induces bronchodilation, resulting in a reduction in the levels of delivery-related coughing. Accordingly, a user is more able to tolerate the composition since it causes very little irritation.

The human TAS2R bitter taste receptor agonist may be a naturally occurring compound or a synthetic compound. Examples of suitable naturally-occurring compounds include Absinthin, Aloin, Amarogentin, Andrographolide, Arborescin, Arglabin, Artemorin, Camphor, Cascarillin, Cnicin, Crispolide, Ethylpyrazine, Falcarindiol, Helicin, Humulone isomers, Limonin, Noscapine Papaverine, Parthenolide, Quassin, Sinigrin, and Thiamine. Examples of suitable synthetic compounds include Acesulfame K, Benzoin, Carisoprodol, Chloroquine, Cromolyn, Dapsone, Denatonium benzoate, Dimethyl thioformamide, Diphenhydramine, Divinylsulfoxide, Famotidine, Saccharin, Sodium benzoate, and Sodium cyclamate.

Preferably the human TAS2R bitter taste receptor agonist is saccharin. Saccharin is particularly effective as a human TAS2R bitter taste receptor agonist, may be readily dissolved in the composition, is readily available and provides the composition with a desirable taste profile. Preferably the formulation comprises 0.001% w/w to 0.1% w/w, more preferably 0.003% w/w to 0.01% w/w and even more preferably 0.005% w/w to 0.008% w/w saccharin. Lower levels of saccharin result in a composition with an unacceptable tolerability. Higher levels of saccharin result in an acceptable tolerability but are disfavoured since saccharin they may lead to precipitates of saccharin forming in the composition, which may cause irritation when the composition is administered to a user or blockage when the composition is incorporated into an inhaler. Such weight percentages also provide the composition with an optimised taste profile.

The composition preferably comprises at least 60% w/w propellant, based on the total weight of the composition, more preferably from 90 to 99.5% w/w, preferably from 92 to 99% w/w, more preferably from 96 to 99% w/w, based on the total weight of the composition. The propellant is preferably liquefied

The composition may further comprise a flavour component. The use of a flavour component may mask the taste of the cannabinoids or derivatives or salts thereof. Suitable flavour components include the flavour components typically added to tobacco products. Examples include carotenoid products, alkenols, aldehydes, esters and delta-lactone flavour constituents. Suitable carotenoid products include beta ionone, alpha ionone, beta-damascone, beta-damascenone, oxo-edulan I, oxo-edulan II, theaspirone, 4-oxo-beta-ionone, 3-oxo-alpha-ionone, dihydroactinodiolide, 4-oxoisophorone, safranal, beta-cyclocitral. Suitable alkenols include C₄ to C₁₀ alkenols, preferably C₅ to C₈ alkenols. Specific examples include: cis-2-Penten-1-ol, cis-2-Hexen-1-ol, trans-2-Hexen-1-ol, trans-2-Hexen-1-ol, cis-3-Hexen-1-ol, trans-3-Hexen-1-ol, trans-2-Hepten-1-ol, cis-3-Hepten-1-ol, trans-3-Hepten-1-ol, cis-4-Hepten-1-ol, trans-2-Octen-1-ol, cis-3-Octen-1-ol, cis-5-Octen-1-ol, 1-Octen-3-ol and 3-Octen-2-ol. Suitable aldehydes include benzaldehyde, glucose and cinnamaldehyde. Suitable esters include allyl hexanoate, benzyl acetate, bornyl acetate, butyl butyrate, ethyl butyrate, ethyl hexanoate, ethyl cinnamate, ethyl formate, ethyl heptanoate, ethyl isovalerate, ethyl lactate, ethyl nonanoate, ethyl valerate, geranyl acetate, geranyl butyrate, isobutyl acetate, isobutyl formate, isoamyl acetate, isopropyl acetate, linalyl acetate, linalyl butyrate, linalyl formate, methyl acetate, methyl anthranilate, methyl benzoate, methyl benzyl acetate, methyl butyrate, methyl cinnamate, methyl pentanoate, methyl phenyl acetate, methyl salicylate (oil of wintergreen), nonyl caprylate, octyl acetate, octyl butyrate, amyl acetate (pentyl acetate), pentyl hexanoate, pentyl pentanoate, propyl ethanoate, propyl isobutyrate, terpenyl butyrate, ethyl formate, ethyl acetate, ethyl propionate, ethyl butyrate, ethyl valerate, ethyl hexanoate, ethyl heptanoate, ethyl octanoate, ethyl nonanoate, ethyl decanoate, ethyl dodecanoate, ethyl myristate, ethyl palm itate. Suitable delta-lactone flavour constituents include delta-Hexalactone, delta-Octalactone, delta-Nonalactone, delta-Decalactone, delta-Undecalactone, delta-Dodecalactone, Massoia lactone, Jasmine lactone and 6-Pentyl-alpha-pyrone. Flavour components may serve to mask the taste of cannabinoids.

The flavour component is preferably menthol and/or vanillin. The presence of menthol, together with the saccharin, reduces the irritation experienced by a user. Preferably the composition comprises up to 0.1% w/w menthol, preferably from 0.01% w/w to 0.08% w/w, more preferably from 0.02% w/w to 0.06% w/w, even more preferably from 0.03% w/w to 0.05% w/w, still even more preferably about 0.04% w/w, based on the total weight of the composition.

The inhalable composition may be for use as a medicament in the treatment of a subject wherein the composition is administered in the form of an aerosol having a fine particle fraction of 60% or more. Such a particle fraction may ensure that the composition is delivered preferentially to lungs. The fine particle fraction is preferably 70% or more, more preferably 75% or more.

The subject may be suffering from a condition selected from: neuropathic pain, cannabis addiction, nausea, motion sickness, arthritis and neurodegenerative diseases such as Alzheimer's, Parkinson's and multiple sclerosis. The subject may be a human.

In a further aspect, the present invention provides an inhalable composition comprising:

one or more cannabinoids or a pharmaceutically acceptable derivative or salt thereof;

a propellant comprising HFA-152a;

a monohydric alcohol; and

a glycol and/or glycol ether.

For the avoidance of doubt, the advantages and preferably features of the first aspect apply equally to this aspect.

The monohydric alcohol (e.g. ethanol) may increase the solubility of the one or more cannabinoids or a pharmaceutically acceptable derivative or salt thereof in the propellant. In addition, the monohydric alcohol (e.g. ethanol) may favourably increase the particle size of the inhalable composition during administration due to a slower rate of evaporation relative to propellant only. However, in view of the high solubility of the one or more cannabinoids or a pharmaceutically acceptable derivative or salt thereof in HFA-152a, such favourable affects may be achieved using lower levels of monohydric alcohol in comparison to conventional inhalable cannabinoid compositions.

The ratio of monohydric or polyhydric alcohol to glycol or glycol ether by weight is preferably from 6:1 to 1:1, preferably from 5:1 to 1.2:1, more preferably from 4:1 to 1.5:1, even more preferably from 3:1 to 2:1. Such ratios may help to provide the favourable droplet sizes referred to above.

The monohydric alcohol is preferably ethanol, which is low cost, easy to handle and widely available.

The composition preferably comprises up to 4% w/w ethanol, based on the total weight of the composition, more preferably up to 3% w/w ethanol, even more preferably up to 1% w/w ethanol, still even more preferably up to 0.5% w/w ethanol, still even more preferably less than 0.5% w/w ethanol, still even more preferably up to 0.4% w/w ethanol. The composition preferably comprises at least 0.01% w/w ethanol, more preferably at least 0.1% w/w ethanol, still even more preferably at least 0.2% w/w ethanol.

In a further aspect, the present invention provides a cannabinoid inhaler comprising:

a housing;

a reservoir within the housing containing the inhalable composition described herein;

a composition flow path from the reservoir and out of a composition outlet at an inhaling end of the housing; and

a non-metered breath operated outlet valve for controlling the flow of inhalable composition through the composition flow path.

The use of a non-metered and breath operated valve provides a number of advantages over the prior art dispensers. As the valve is breath operated, it can only be opened when a user is inhaling such that, at the moment the valve opens to release the composition, there is an existing flow of air into the lungs thereby facilitating the entrainment of the composition into the lungs. Further, as the administration of formulation is unmetered, the user can self-titrate and can obtain a dose (i.e. one fill of the device) over a number of inhalations (or puffs) at a desired, comfortable pace. This can be done in a few inhalations and potentially in a single inhalation if desired.

Thus, the present invention provides a cannabinoid inhaler, which is easy to use and provides a way of obtaining a relatively consistent dose with minimal wastage for deep lung administration and rapid onset of clinical actions as compared to a metered dose inhaler or spray.

The breath-operated valve may have a number of configurations. It may, for example, comprise an electronic flow sensor which detects suction on the inhaling end and activates a solenoid to open the outlet valve. Alternatively, the valve may be a heater which selectively vaporises a proportion of a viscous composition, with the reservoir being configured to replenish the viscous composition in the vicinity of the heater. As a further example, the valve may take the form of a Venturi nozzle which generates a suction force when a user inhales. The suction force may directly remove the composition from the housing, or the valve may further comprise a closure element which is opened by the suction force.

The important consideration for the outlet valve is that it is able to selectively allow the dispensing of composition in response to a user inhaling from the inhaler.

The breath operated inhaler preferably further comprises an air flow path from an inlet spaced from the inhaling end of the inhaler to an air outlet at the inhaling end, the air flow path being configured such that suction on the inhaling end causes flow through the air flow path which causes the breath operated valve to open, the air outlet being positioned adjacent to the composition outlet, such that air from the air outlet impinges on the composition leaving the composition outlet.

The provision of an air flow path which both operates the breath operated valve and also impinges on the composition outlet provides a dual benefit in opening the valve and having a beneficial effect on the particle size, as the impinging air breaks up larger droplets of the composition thereby significantly decreasing the mean particle size. If an air flow outlet is provided on either side of the inhalable composition outlet, this effect is enhanced and any deflection of the composition plume caused by a single air outlet is avoided.

The air flow outlets are preferably directed towards a composition plume emitted, in use, from the inhaler to break up the particle size of the composition plume.

The outlet valve preferably comprises:

a flexible diaphragm within the housing and being positioned so as to be influenced by the air flowing through the air flow path;

a valve element movable with the diaphragm and biased by a biasing force into a position in which it closes the composition flow path;

wherein suction on the inhaling end causes a flow through the air flow path providing a pressure differential across the diaphragm thereby lifting the valve element against the biasing force to open the composition flow path; and wherein the biasing force is arranged to close the composition flow path once the suction ceases.

The use of a flexible diaphragm is beneficial as it can provide a relatively large surface area so that it is able to open the valve even with a relatively low flow rate. This allows the inhaler to open at a relatively low flow rate as compared to a standard metered dose inhaler, which is useful for patients who find it difficult to inhale deeply.

The inhaler preferably further comprises a first air flow path partly defined by one side of the diaphragm, a second air flow path partly defined by the opposite side of the diaphragm, each flow path having an opening at the outlet end, wherein the air flow paths are arranged such that suction at the outlet end results in a pressure differential across the diaphragm that moves the diaphragm and hence moves the valve element against the biasing force to open the composition flow path. The presence of the first and second air flow paths minimises further the suction required to open the valve.

Preferably, at least a portion of the flow path is a deformable tube, and the outlet valve is provided by a clamping member which pinches the deformable tube closed when no suction force is applied to the inhaling end to close the composition flow path and releases the tube to open the composition flow path when suction is applied at the inhaling end. The deformable tube provides a simple mechanism for the breath-operated valve which operates particularly well with the flexible diaphragm to provide a simple, reliable and easy to operate breath-operated valve.

In one preferred embodiment there is a single diaphragm and valve element.

The inhaler preferably further comprises a tube having an internal bore leading from an inlet on a main axis of the housing a reservoir outlet. The inlet of the bore is preferably in the vicinity of the centroid of the volume of the reservoir.

The inhaler may be designed for a single use. However, preferably, the inhaler has a refill valve in communication with the reservoir via which the reservoir may be refilled.

The reservoir may be at atmospheric pressure. However, it is preferably pressurised as this pressure can provide the motive force to expel the composition from the reservoir. The reservoir may be pressurised by using a compressed gas. However, preferably, the formulation further comprises a propellant as this allows the pressure in the reservoir to be substantially maintained as the composition is dispensed.

Preferably, the pressure within the reservoir and the size of the composition flow path at its narrowest point are arranged so that, when the outlet valve is fully opened, the reservoir will discharge in less than 30 seconds.

Such a reservoir is an optimal size for cannabinoid delivery as it allows a user to obtain a dose over 8 to 10 puffs, on average. However, the inhaler is not so large that it contains a dose which is likely to be harmful to either the authorised patient or some third party. In view of this, it is not necessary to provide a lock-out mechanism on the inhaler itself.

Preferably, the inhaler is configured to eject inhalable composition therefrom in the form of droplets, at least some of which have a diameter of 10 μm or less, and preferably at least 99% vol of the droplets have a diameter of less than 10 μm.

This relatively small particle size is ideal for pulmonary delivery, and co-operates particularly well with a breath-operated valve to ensure that there is a flow of relatively small particle size delivered into the pre-existing suction air stream ensuring even more reliable and repeatable delivery deep into the lungs.

In a further aspect, the present invention provides a pressurised container containing the composition as described herein. The pressurised container may be used to release a gaseous flow of the composition to a user. For example, the pressurised container may be provided with means for delivering the contents of the container to the lungs of a user. Such means may take the form of a button, trigger or breath-activated mechanism. The pressurised container may be used to deliver an unmetered dose of cannabinoid to the user. This may be advantageous over prior art methods of cannabis replacement therapy, such as conventional inhalers, nasal sprays, lozenges and patches currently on the market, because it can allow autonomy in cannabinoid replacement regulation, where the user can regulate the amount of compositional cannabinoid he or she wishes to inhale. In addition, the pressurised container can be used as an alternative to recreational smoking of conventional cannabis-containing cigarettes.

The pressurised container of the present invention may be used to release the composition to a user without the need for a separate source of energy. For example, the composition may be released without requiring the heating of substrates, combustion of material or a battery powered electric current. As discussed above, this can result in a reduction in the levels of harmful by-products delivered to a user.

The pressurised container of the present invention may take the form of a pressurised canister, for example, a pressurised aluminium canister. The canister may be fully recyclable and/or reusable. The canister may be refilled as required by a vending machine or a larger container containing the desired composition under a high pressure gradient. In one embodiment, the canister is an AW5052 aluminium canister.

The pressurised container may be capable of dispensing the composition as a mixture of aerosolised droplets. Preferably, the mixture has a particle size distribution that is similar to tobacco smoke. The mixture may have the appearance of a vapour or smoke.

The pressurised container may be pressurised to a pressure of from 3×10⁵ Pa to 1.5×10⁷ Pa, preferably from 5×10⁵ Pa to 2×10⁶ Pa, more preferably from 5.5 ×10⁵ Pa to 1×10⁶ Pa, even more preferably at about 6 x 10⁵ Pa.

In a futher aspect, the present invention provides a method of manufacturing the composition described herein, the method comprising:

preparing a pre-mixture comprising a glycol or glycol ether, and optionally a monohydric alcohol, a TAS2R taste receptor agonist and/or flavouring component;

adding one or more cannabinoids or pharmaceutically acceptable derivatives or salts thereof, to the pre-mixture to obtain a cannabinoid-containing mixture; and

adding a propellant to the cannabinoid-containing mixture, wherein the propellant comprises HFA-152a.

If the cannabinoid is added before the polyhydric alcohol and glycol or glycol ether are combined, then precipitation of cannabinoid (and, if present, nicotine) may occur. Likewise, if the composition comprises other components, such as a flavouring component or a TAS2R taste receptor agonist, then these components should be fully mixed into the pre-mixture before the cannabinoids (and optionally nicotine) is added in order to avoid precipitation of cannabinoids (and, if present, nicotine). In particular, it has been found that when the composition comprises menthol, the menthol should be fully dissolved into the pre-mixture before the cannabinoids (and optionally nicotine) is added in order to avoid precipitation of the cannabinoids (and, if present, nicotine).

When the composition comprises a TAS2R taste receptor agonist and/or flavouring component, preferably the glycol or glycol ether and optionally the alcohol are combined before the TAS2R taste receptor agonist and/or flavouring component are added. This avoids precipitation of the flavouring component or TAS2R taste receptor agonist.

### BRIEF DESCRIPTION OF THE DRAWINGS

FIG. 1 is an exploded perspective view of an inhaler;

FIG. 2 is a schematic axial cross-section through the outlet end of the inhaler in the plane containing an air flow path and with the vane removed for clarity;

FIG. 3 is a perspective view of the outlet end of the inhaler with the cover, vane and diaphragm removed to show the air flow paths;

FIG. 4 is a perspective view of the outlet end of the inhaler;

FIG. 5 is a plan view of the inhaler;

FIG. 6 is a full cross-section of the inhaler;

FIG. 6A is a cross-section through line 6A-6A in FIGS. 6; and

FIGS. 7-9 are cross-sectional views of an inhaler of a second example in various orientations.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

The inhaler described below is based on that disclosed in WO 2010/073018. For further details of the device and its refill mechanism, reference is made to WO 2009/001078 and WO 2011/095781.

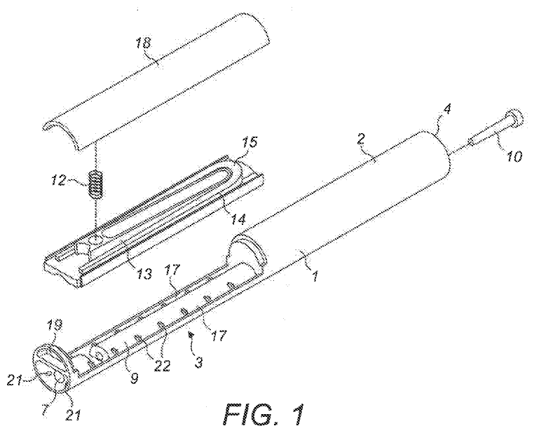

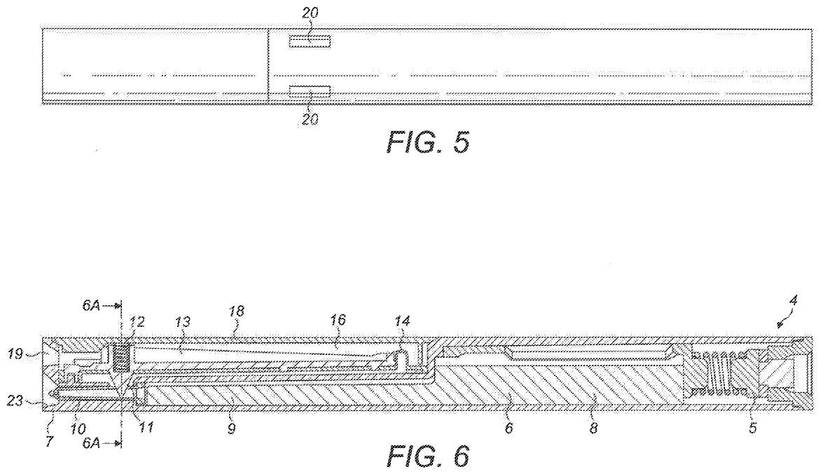

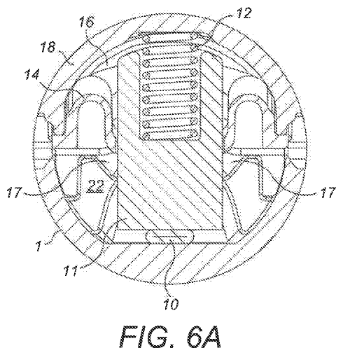

As shown in FIG. 1, the inhaler comprises a housing 1 which is broadly divided into two parts. The distal part is a reservoir 2 and the proximal part is the breath-activated valve mechanism 3. At the refill end 4 is a refill valve 5 allowing the reservoir to be filled. The reservoir may contain a wick 6 as shown in FIG. 6 and disclosed in WO 2011/107737. At the opposite end is the outlet end 7 which will be described in more detail below.

An elastomeric insert 10 (described in greater detail in GB 1305496.0) in the form of a tube open at both ends is inserted from the distal end. This insert 10 is normally pinched closed by a valve element 11 which is biased downwardly by a spring 12. This pinch closed valve mechanism is described in greater detail in WO 2011/015825.

The valve element 11 is part of a vane 13 which extends along most of the outlet end of the inhaler. The vane 13 is surrounded by a diaphragm 14 which extends across the entire lower face of the vane 13, with the exception of the orifice through which the valve element 11 projects. This valve element is sealed around its periphery to the surrounding housing. At the distal end of the diaphragm 14 is a kink 15 which provides some degree of freedom for the vane 13 to move up and down. The opposite end of the vane 13 is integral with a surrounding frame that is fitted into the housing such that there is a direct connection between the frame and vane to provide a hinge about which the vane pivots.

A mechanism for opening the valve element 11 against the action of the spring 12 will now be described.

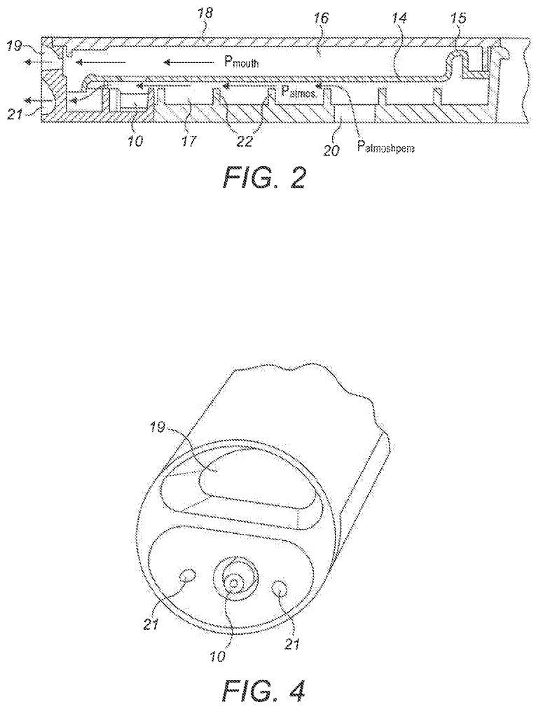

This is achieved by first 16 and second 17 air flow paths as best shown in FIG. 2. The first flow path 16 is above the diaphragm 14 with the top of the flow path being formed by housing part 18 which is fixed to the housing 1 once the valve elements are in place. The first air flow path is essentially provided by a first air flow path outlet orifice 19 which leads into the space occupied by the vane 13 above the diaphragm 14. This flow path has no other orifices.

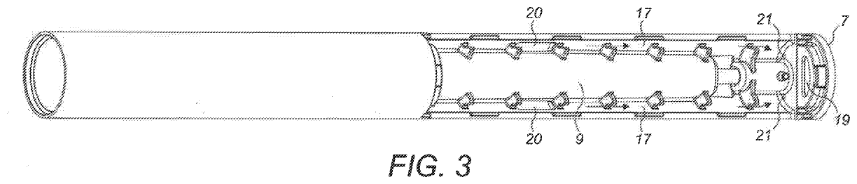

The second air flow path 17 is below the diaphragm 14 and is defined by a pair of second air flow path inlet orifices 20 (only one of which is shown in FIG. 2). In the present example, the second air flow path is actually defined by two separate paths which extend from the inlet orifices 20 along passages 17 which are defined by the housing 1 on the lower surface and the diaphragm 11 at its upper surface and which extends alongside the second portion 9 of the reservoir to the outlet end terminating at a pair of second air flow path outlet orifices 21 which are smaller than the corresponding inlet orifices 20 and are directed towards it to break up the particle size of the composition plume as described in greater detail in GB 1215282.3. The flow through the second air flow path is depicted by arrows in the lower part of FIG. 2 and in FIG. 3. Baffles 22 are provided along the second air flow path 17 to increase the flow resistance in this path.

As a user sucks on the outlet end 7, air is sucked out of the first flow path outlet orifice 19 thereby lowering the pressure in the first air flow path 16. At the same time, air is drawn in through the second flow path air inlet orifices 20. As these are larger than the second flow path outlet orifices 21, a choking effect aided by the baffles 22 effectively causes pressure to increase in the second air flow path. A combination of a reduced pressure above the vane and a raised pressure below the vane 13 causes the vane to be moved upwardly deforming the diaphragm 14 and raising the valve element against the action of the spring 12. When a user stops sucking on the outlet end 7, the pressure above and below the diaphragm 14 equalises and the spring 12 returns the valve element 11 to a position in which it pinches the insert 10 closed.

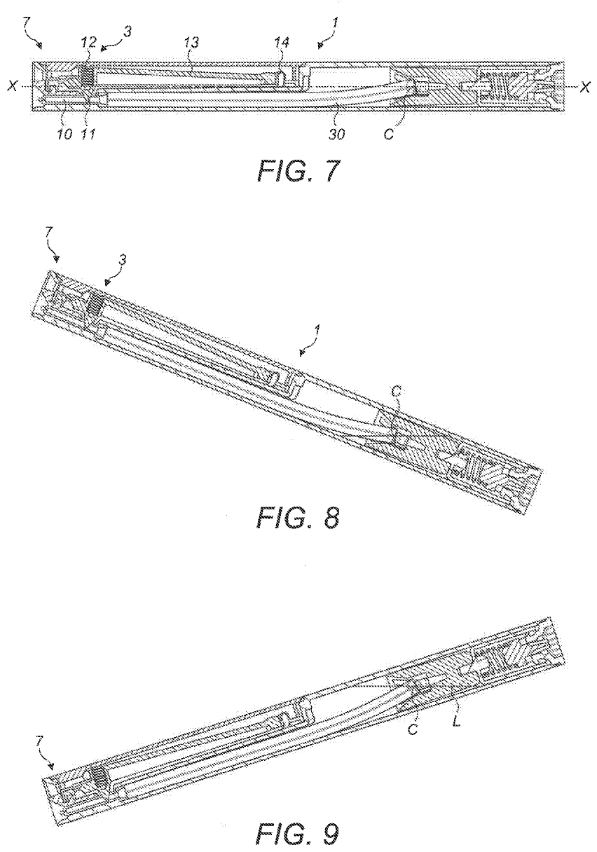

A second example of an inhaler is shown in FIGS. 7 to 9. This is described in greater detail in GB 1305494.5. In place of the wick 6, this example is provided with a tube 30 having an internal bore 31 leading to the insert 10 at the opposite end of tube 31.

At the inlet end 32 of the tube 30, the bore 31 has an inlet 33 which is supported by a support 34 so that the inlet end 32, and preferably the inlet 33 of the bore 31 is on the main axis X of the housing 1 as shown in FIG. 7.

It will be appreciated from the drawings that the shape of the reservoir is complex. The right hand portion has a generally cylindrical configuration occupying the majority of the diameter of the device while the left hand portion of the reservoir may just be the internal bore 31 of the tube, or there may be a portion of the reservoir on either side of this tube. Further, in the right hand portion, the volume of the reservoir is reduced by the inlet end portion of the tube 30, the support 34, and the refill valve assembly 5. Thus, while the volume of the reservoir 4 can be determined by measuring these components, it may be simpler to determine this experimentally.

The operation of the device will now be described with reference to FIGS. 7 to 9.

When a user sucks on the outlet end 7, the outlet valve 3 opens as previously described. Provided that the inlet 33 of the bore 31 is below the level L of the liquid in the reservoir, the liquid will travel along the bore 31 and will be atomised downstream of the outlet valve element 11 to create a plume for inhalation. FIGS. 7 to 9 show the centroid C of a body of liquid filling the reservoir 4. The inlet 33 of the bore 31 is in the vicinity of the centroid. In this specific example shown in FIG. 1, it is displaced by 1.3 mm from the centroid C towards the refill end 4. In the horizontal orientation shown in FIG. 1, all of the liquid above the level L which represents approximately 50% of the total liquid in the reservoir can be inhaled from the inhaler. When the inhaler is in the tip-down configuration shown in FIG. 2, as the inlet 33 is displaced from the centroid C as described above, slightly more liquid is available than it is in FIG. 1. Conversely, in the tip-up configuration, slightly less liquid is available for inhalation. In a different arrangement, the inlet 33 is at the centroid C, so that there is essentially no variation in dispensing between the three positions. The current preference is for a slight displacement of the inlet 33 towards the refill end from the centroid C as shown as this causes slightly more liquid to be dispensed in the more common tip-down orientation.

Once the cigarette reaches the liquid level position L shown in FIGS. 7 to 9 with the reservoir approximately half full, no further liquid can be inhaled and the inhaler then needs to be refilled via the refill valve 5.

The foregoing detailed description has been provided by way of explanation and illustration, and is not intended to limit the scope of the appended claims. Many variations in the presently preferred embodiments illustrated herein will be apparent to one of ordinary skill in the art and remain within the scope of the appended claims and their equivalents.

1. An inhalable composition comprising:
one or more cannabinoids or a pharmaceutically acceptable derivative or salt thereof;
a propellant comprising HFA-152a; and
at least one of a glycol and a glycol ether.

2. The inhalable composition of claim 1, wherein the composition comprises less than 1% w/w ethanol.

3. The inhalable composition of claim 1, wherein the composition comprises less than 0.5% w/w ethanol.

4. The inhalable composition according to claim 1, wherein the composition is free of ethanol.

5. The composition according to claim 1, wherein the one or more cannabinoids is selected from tetrahydrocannabinol (THC), preferably dronabinol, cannabidiol (CBD), cannabinol (CBN), tetrahydrocannabivarin (THCV), cannabigerol (CBG), cannabidivarin (CBDV) and cannabichromene (CBC).

6. The composition according to claim 1, wherein the one or more cannabinoids is dronabinol.

7. The composition according to claim 1 comprising from 0.01 to 15% w/w of the one or more cannabinoids or pharmaceutically acceptable derivatives or salts thereof based on the total weight of the composition.

8. The composition according to claim 1, wherein the at least one of the glycol and the glycol ether is selected from the group consisting of propylene glycol, polypropylene glycol, polyethylene glycol (PEG), and combinations of two or more thereof.

9. The composition according to claim 1, wherein the at least one of the glycol and the glycol ether is propylene glycol and the composition comprises from 0.01 to 5% w/w propylene glycol, based on the total weight of the composition.

10. The composition according to claim 1, wherein the at least one of the glycol and the glycol ether is propylene glycol and the composition comprises from 0.1 to 2% w/w propylene glycol, based on the total weight of the composition.

11. The composition according to claim 1, wherein the composition further comprises a human TAS2R bitter taste receptor agonist.

12. The composition according claim 1, wherein the composition further comprises saccharin in an amount by weight from 0.001% w/w to 0.1% w/w.

13. The composition according claim 1 comprising at least 60% w/w propellant, based on the total weight of the composition.

14. The composition according claim 1 further comprising a flavour component, preferably selected from at least one of peppermint oil, aniseed, chocolate, coco, menthol, and vanillin.

15. The composition according to claim 14, wherein the composition comprises up to 0.1% w/w menthol, based on the total weight of the composition.

16. The inhalable composition of claim 1 for use as a medicament in the treatment of a subject wherein the composition is administered in the form of an aerosol having a fine particle fraction of 60% or more.

17. The composition for use according to claim 16, wherein the fine particle fraction is 70% or more.

18. The composition for use according to claim 16, wherein the fine particle fraction is 75% or more.

19. The composition for use according to claim 16, wherein the subject is suffering from a condition selected from:
neuropathic pain, cannabis addiction, nausea, motion sickness, arthritis and neurodegenerative diseases such as Alzheimer's, Parkinson's and multiple sclerosis.

20. The composition for use according to claim 16, wherein the subject is a human.

21. An inhalable composition comprising:
one or more cannabinoids or a pharmaceutically acceptable derivative or salt thereof;
a propellant comprising HFA-152a;
a monohydric alcohol; and
at least one of a glycol and a glycol ether.

22. The inhalable composition of claim 21, wherein the ratio of monohydric or polyhydric alcohol to glycol or glycol ether by weight is from 6:1 to 1:1.

23. The composition according to claim 21, wherein the monohydric alcohol is ethanol.

24. The composition according to claim 23 wherein the composition comprises from 0.01 to 0.5% w/w ethanol, based on the total weight of the composition.

25. A cannabinoid inhaler comprising:
a housing;
a reservoir within the housing containing an inhalable composition, wherein the inhalable composition includes:
one or more cannabinoids or a pharmaceutically acceptable derivative or salt thereof,
a propellant comprising HFA-152a, and
at least one of a glycol and s glycol ether;
a composition flow path from the reservoir and out of a composition outlet at an inhaling end of the housing; and
a non-metered breath operated outlet valve for controlling the flow of inhalable composition through the composition flow path.

26. The inhaler according to claim 25, wherein the breath operated inhaler further comprises an air flow path from an inlet spaced from the inhaling end of the inhaler to an air outlet at the inhaling end, the air flow path being configured such that suction on the inhaling end causes flow through the air flow path which causes the breath operated valve to open, the air outlet being positioned adjacent to the composition outlet, such that air from the air outlet impinges on the composition leaving the composition outlet.

27. The inhaler according to claim 26, wherein there is a respective air flow outlet on either side of the inhalable composition outlet.

28. The inhaler according to claim 27, wherein the air flow outlets are directed towards a composition plume emitted, in use, from the inhaler to break up the particle size of the composition plume.

29. The inhaler according to claim 25 wherein the outlet valve comprises:
a flexible diaphragm within the housing and being positioned so as to be influenced by the air flowing through the air flow path;
a valve element movable with the diaphragm and biased by a biasing force into a position in which it closes the composition flow path;
wherein suction on the inhaling end causes a flow through the air flow path providing a pressure differential across the diaphragm thereby lifting the valve element against the biasing force to open the composition flow path; and
wherein the biasing force is arranged to close the composition flow path once the suction ceases.

30. The inhaler according to claim 29, further comprising a first air flow path partly defined by one side of the diaphragm, a second air flow path partly defined by the opposite side of the diaphragm, each flow path having an opening at the outlet end, wherein the air flow paths are arranged such that suction at the outlet end results in a pressure differential across the diaphragm that moves the diaphragm and hence moves the valve element against the biasing force to open the composition flow path.

31. The inhaler according to claim 25, wherein at least a portion of the flow path is a deformable tube, and the outlet valve is provided by a clamping member which pinches the deformable tube closed when no suction force is applied to the inhaling end to close the composition flow path and releases the tube to open the composition flow path when suction is applied at the inhaling end.

32. The inhaler according to claim 25, wherein there is a single diaphragm and valve element.

33. The inhaler according to claim 25, further comprising tube having an internal bore leading from an inlet on a main axis of the housing a reservoir outlet.

34. The inhaler according to claim 33, wherein the inlet of the bore is in the vicinity of the centroid of the volume of the reservoir.

35. The inhaler according to claim 25, further comprising a refill valve in communication with the reservoir via which the reservoir may be refilled.

36. The inhaler according to claim 25, wherein the reservoir is pressurised, preferably wherein the inhalable composition further comprises a propellant.

37. The inhaler according to claim 36, wherein the size of the reservoir, the pressure within the reservoir, and the size of the composition flow path at its narrowest point are arranged so that, when the outlet valve is fully opened, the reservoir will discharge in less than 30 seconds.

38. The inhaler according to claim 25, wherein the inhaler is configured to eject the inhalable composition therefrom in the form of droplets, at least 99% vol of the droplets having a diameter of less than 10 microns.

39. A pressurised container containing a composition consisting of:
one or more cannabinoids or a pharmaceutically acceptable derivative or salt thereof;
a propellant comprising HFA-152a; and
at least one of glycol and a glycol ether.

40. The pressurised container of claim 39 pressurised to a pressure of from 3×10⁵ Pa to 1.5×10⁷ Pa.

41. A method of manufacturing a composition, the method comprising:
preparing a pre-mixture comprising a glycol or glycol ether, and at least one of a monohydric alcohol, a TAS2R taste receptor agonist, and flavouring component;
adding one or more cannabinoids or pharmaceutically acceptable derivatives or salts thereof, to the pre-mixture to obtain a cannabinoid-containing mixture; and
adding a propellant to the cannabinoid-containing mixture, wherein the propellant comprises HFA-152a.

42. The method according to claim 41, wherein the composition comprises at least one of the TAS2R taste receptor agonist and flavouring component, and wherein the glycol or glycol ether are combined before at least one of the TAS2R taste receptor agonist and flavouring component are added.
